(19) United States
(12) Patent Application Publication
(10) Pub. No.: US 2025/0050003 A1
(43) Pub. Date: Feb. 13, 2025

(54) PNEUMATICALLY DRIVEN PERITONEAL DIALYSIS MACHINE

(71) Applicants: BAXTER INTERNATIONAL INC., Deerfield, IL (US); BAXTER HEALTHCARE SA, Glattpark (Opfikon) (CH)

(72) Inventors: Karthik PITCHAIMANI, Bangalore, Karnataka (IN); Anoop Thirumattathil ASHOKAN, Bangalore, Karnataka (IN)

(21) Appl. No.: 18/722,847

(22) PCT Filed: Dec. 6, 2022
(86) PCT No.: PCT/US2022/080973

§ 371 Date: Jun. 21, 2024

(30) Foreign Application Priority Data
Dec. 21, 2021 (IN) 202141059786

(51) Int. Cl. A61M 1/28

(52) U.S. Cl. CPC A61M 1/282; A61M 2205/3331

(57) ABSTRACT

A peritoneal dialysis (“PD”) system includes a disposable set including a pump chamber having a flexible sheet, one side of the flexible sheet positioned and arranged during operation to receive pneumatic pressure; and a cycler including at least one source of positive and negative pneumatic pressure for delivering pneumatic pressure to the pump chamber, an air flow sensor, a pneumatic pressure sensor, a temperature sensor, a plurality of fluid valves, and a control unit configured to integrate outputs from the air flow sensor, the pneumatic pressure sensor and the temperature sensor over time to determine an amount of fresh or used PD fluid discharged from the pump chamber under positive pneumatic pressure and via an open one of the plurality of fluid valves.

### PRIORITY CLAIM

The present application claims priority to and the benefit of Indian Provisional Application No. 202141059786, filed on Dec. 21, 2021, the entire contents of which are hereby incorporated by reference.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the peritoneal chamber, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities using an automated machine, the automated machine may be operated pneumatically. Pneumatic systems operated using the Ideal Gas Law have typically required a reference chamber to enable before and after pump stroke air volume calculations to be made. The volume of fluid pumped over the stroke is then the difference between the before and after air volumes. The reference chamber adds cost and consumes space. A valve is also required between the pump chamber and the reference chamber, which also adds cost and requires sequencing for each pump stroke.

For each of the above reasons, it is desirable to provide an improved pneumatically driven APD machine or cycler.

### SUMMARY

The present disclosure sets forth an automated peritoneal dialysis (“APD”) system having a machine or cycler that operates with a disposable set having a pump chamber, such as a pod pump chamber. In one possible configuration for the system, the disposable set includes multiple peritoneal dialysis fluid containers or bags, wherein one of the containers is placed on top of the cycler, which includes a heating plate to heat dialysis fluid located originally in the container as well as dialysis fluid pumped to the container from a second or later container for a subsequent patient fill. In an alternative embodiment, the plate or batch heater is replaced with an inline heater, which heats fresh dialysis fluid as it flows through the patient line to the patient. The disposable pump chamber may be oriented vertically as illustrated herein, wherein fluid tubes or lines run horizontally from the pump chamber. The disposable pump chamber may alternatively be oriented horizontally.

An air pump for driving the disposable pump chamber and other reusable components herein may be located within a housing of the machine or cycler. The air pump may or may not operate with pneumatic storage vessels that store positive and negative pneumatic pressure. The storage vessels if provided are used to deliver positive and negative pneumatic pressure to the pump chamber. If the storage vessels are not provided, the air pump is configured to provide both positive and negative pressure air directly to the disposable pump chamber via pneumatic lines. The pneumatic lines include a pneumatic suction line and a pneumatic delivery line. Each of the pneumatic suction line and the pneumatic delivery line includes or is provided with a pneumatic valve that either opens or closes the pneumatic line to atmosphere. The pneumatic suction line and the pneumatic delivery line meet at a common pneumatic line that extends to a reusable pump actuation chamber.

To apply negative pneumatic pressure to the pump actuation chamber, the pneumatic valve located along the pneumatic delivery line is set to vent, while the pneumatic valve located along the pneumatic suction line is closed to atmosphere. The air pump is actuated to create a set negative pneumatic pressure in the pneumatic suction and common lines, which pulls on a flexible pumping sheet, diaphragm or membrane of the disposable pump chamber, which is coupled against the reusable pump actuation chamber. Air pulled to create the negative pneumatic pressure is vented to atmosphere via the pneumatic valve located along the pneumatic delivery line.

To apply positive pneumatic pressure to the pump actuation chamber, the pneumatic valve located along the pneumatic suction line is set to vent, while the pneumatic valve located along the pneumatic delivery line is closed to atmosphere. The air pump is actuated (e.g., in the same direction as above) to create a set positive pneumatic pressure in the pneumatic delivery and common lines, which pushes on the flexible pumping sheet, diaphragm or membrane of the disposable pump chamber, which is again coupled against the reusable pump actuation chamber. Air used to create the positive pneumatic pressure is pulled in via the pneumatic valve along the pneumatic suction line.

In an embodiment, multiple sensors are located along the common pneumatic line, including an air flow sensor, a pneumatic pressure sensor and a pneumatic temperature sensor. The outputs from each of the air flow, pressure and temperature sensors are used for the Ideal Gas Law fluid volume determinations discussed below. The output from the pressure sensor is also used as feedback for the control of the air pump to deliver Peritoneal Dialysis (“PD”) fluid to and from the patient at desired positive and negative pressures, respectively.

The disposable set may include five fluid lines that extend from the disposable pump chamber, including three lines extending to three PD fluid containers or bags in one embodiment, one of which sits atop a batch heater as mentioned above. The third PD fluid container or bag may be provided with a last fill formulation of PD, such as icodextrin. The fourth line may be a drain line extending to a drain container or bag or to a house drain (e.g., toilet or bathtub). The fifth line is a patient line.

Each of the five fluid lines may be fitted into or operate with a pinch valve, which may be an electrically actuated solenoid valve. The pinch valves are fail safe in one embodiment, meaning that upon power loss the valves are biased to close their respective fluid lines. The valves may alternatively be pneumatically operated valves, such as pillow valves or cassette-based valves.

For priming, it is contemplated that the PD machine or cycler provide a prime sensor/bubble detector, e.g., an optical or capacitive sensor, which operates with the patient line to look for (i) fluid during priming to know that the patient line has been fully primed and (ii) air during treatment. If air is detected during treatment, the air-entrained PD fluid may be pulled back into the disposable pump chamber and then pumped out to drain.

The disposable pump chamber if implemented as a disposable pod pump chamber may be constructed in multiple ways. In one embodiment the pod pump chamber includes a rigid, e.g., plastic disposable shell and the flexible sheet, diaphragm or membrane mentioned above fixed, e.g., ultrasonically welded, to the shell. Here, positive and negative pneumatic pressure is supplied from the air pump and the common pneumatic line to the flexible sheet, diaphragm or membrane. In another embodiment, the disposable pod pump chamber includes two rigid plastic shells, namely, a pneumatic rigid plastic shell and a fluid contacting rigid plastic shell, which are sealed together to hold the flexible sheet, diaphragm or membrane in a sealed manner therebetween. A central pneumatic port is provided in the pneumatic rigid plastic shell, which communicates pneumatically with the common pneumatic line and the air pump. Here, air resides between the pneumatic rigid plastic shell and the flexible membrane. In either of the above embodiments, five fluid ports extend from the fluid contacting rigid plastic shell, which connect to the five fluid lines discussed above. Fresh, fresh heated, or used PD fluid resides accordingly between the fluid contacting rigid plastic shell and the flexible membrane. In either embodiment, the flexible membrane is (i) pulled towards the common pneumatic line under negative pressure to pull fresh or used dialysis fluid into the disposable pod pump chamber, and (ii) pushed away from the common pneumatic line under positive pressure to push fresh or used dialysis fluid from the disposable pod pump chamber.

The pump chamber, the flexible plastic sheet, the fluid lines and fluid containers of the disposable set may be made of one or more plastic, e.g., polyvinylchloride (“PVC”) or a non-PVC material, such as polyethylene (“PE”), polyurethane (“PU”) or polycarbonate (“PC”). The housing of the cycler may be made of any of the above plastics, and/or of metal, e.g., stainless steel, steel and/or aluminum. The housing of the cycler may take different forms, e.g., the user interface may rotate up or out from the housing or may be integrated with the housing.

A control unit having one or more processor, one or more memory and a video controller operating with a user interface is provided to control each of the fluid valves, each of the pneumatic valves, the air pump, and the heater and to receive signals from each of the air flow sensor, pressure sensor, temperature sensor, and priming sensor or air detector. The user interface may be provided with a touchscreen and/or electromechanical pushbuttons to allow the user or patient to enter parameters for treatment and a display screen for providing information, such as treatment status information. The control unit is also programmed to perform the following calculations to determine how much fresh or used PD fluid has been pumped by the disposable pump chamber.

In one embodiment, the control unit is programmed to cause (i) approximate PD fluid volume measurements to be made for anytime that fresh or used PD fluid is drawn into the pump chamber (is under negative pneumatic pressure) and (ii) accurate PD fluid volume measurements to be made for anytime that fresh or used PD fluid is discharged from the pump chamber (is under positive pneumatic pressure). The approximate PD fluid volume measurements made for drawing PD fluid are accurate enough to determine if an occlusion exists in a line or if a currently used PD fluid container or bag is at or near empty. The accurate PD fluid volume measurements made for discharging PD fluid are accurate enough to ensure (i) that prescribed patient fill volumes are met and (ii) that patient drain volumes are accurate so that an accurate determination of the amount of ultrafiltration (“UF”) removed from the patient may be determined. The accurate PD fluid volume measurements made for discharging PD fluid are also accurate enough to determine line occlusions and to confirm container or bag empty conditions based on summed stroke volumes.

In an embodiment, the control unit performs the accurate (discharging) PD fluid volume measurements by closing all fluid valves and causing the air pump to pressurize the pneumatic side of the flexible sheet to a specified pressure, e.g., five psig. The control unit next causes a desired discharge fluid valve to open, allowing the five psig pneumatic pressure to move the flexible sheet, in turn causing fresh or used PD fluid to be discharged from the pump chamber. At this time, air also moves past the air flow sensor at varying pressures. In an optional embodiment, during the discharge of PD fluid, the control unit causes the air pump, under feedback control from the pressure sensor, to pressurize the pneumatic side of the flexible sheet to a specified pressure, e.g., at or just below five psig, which reduces or virtually eliminates repressurization time after the discharge of fresh or used PD has been completed. As mentioned herein, during the discharge of PD fluid, the control unit in an embodiment looks at the resulting flowrate profile to determine if a partial or complete occlusion or a bag empty condition has occurred. The control unit alternatively or additionally determines an occlusion or empty condition based on a measured PD fluid discharge volume compared against an expected PD fluid discharge volume.

The outputs from the air flow sensor, pneumatic pressure sensor and temperature sensor located along the common line are recorded by the control unit over multiple, e.g., many, points in time while the desired discharge fluid valve is open. After a set period of time, the control unit causes the desired discharge fluid valve to close. Next, the control unit, if and to the extent needed based on the repressurization discussed above, causes the air pump to repressurize the pneumatic side of the flexible sheet to the specified pressure, e.g., five psig.

At the end of each of time period in which air flow sensor, pressure sensor and temperature sensor data is being continuously monitored and recorded, the control unit feeds the data into an algorithm that determines the amount of fresh or used PD fluid discharged from the pump chamber over that time period. The algorithm is in one embodiment:

$$Δ V = \frac{[ \sum P ( t ) \frac{f ( t ) Δ t}{RT ( t )} ]}{P} RT ,$$

which is derived from

$$Δ V = \frac{\sum dn}{P} RT ,$$

which is derived from the Ideal Gas Law, and wherein R is a constant, f for a given point in time is the measured flowrate, P for the given point in time is the measured pressure and T for the given point in time is the measured temperature. In particular,

∫fdt≅f(t)Δt,
such that at each sampled data point, Δt=1/sampling rate, Hz, and f, P, T are measured values at that sampled data point time.

The control unit next sums the amounts of fresh or used PD fluid discharged from the pump chamber over each time period of a full discharge stroke to determine a stroke discharge volume. The control unit then sums each of the stroke discharge volumes to determine a fresh PD fluid fill volume delivered to the patient or a used PD fluid drain volume removed from the patient. The control unit may be programmed to stop incrementing a fresh PD fluid fill volume when it meets a prescribed PD fluid fill volume. The control unit may be programmed to stop incrementing a used PD fluid drain volume when it meets a prescribed PD fluid drain volume or when a flowrate of used PD fluid drops to a point indicating that the patient is effectively empty.

In an embodiment, the control unit performs the approximate PD fluid volume measurements for drawing fresh or used PD fluid into the pump chamber by closing all fluid valves and causing the air pump to vent the pump actuation chamber to atmosphere via the pneumatic valve located along the pneumatic delivery line being set to vent. Next, the control unit with all fluid valves still closed causes the air pump to pressurize the pneumatic side of the flexible sheet (pump actuation chamber) to a specified pressure, e.g., five psig. At this step, air moves past the air flow sensor at varying pressures. The outputs from the air flow sensor, pressure sensor and temperature sensor located along the common line are recorded by the control unit over multiple points in time until the measured pressure reaches a specified value, e.g., five psig on the pneumatic side of the flexible sheet. Because the fluid side valves are closed, the fluid side pressure follows the pneumatic side pressure over time.

The control unit next causes the air pump to apply a desired draw pressure, e.g., negative three psig pneumatic pressure, and a desired intake fluid valve to open to move the flexible sheet, in turn causing fresh or used PD fluid to be drawn into the pump chamber. The control unit does not take realtime flowrate, pressure and temperature measurements during the actual fresh or used PD fluid draw as part of the approximation. The control unit instead performs a before and after evaluation as shown below. After a specified amount of time, which may be an amount of time expected to fully fill the pump chamber, the control unit causes the intake fluid valve to close. In an optional embodiment, the air flow sensor is bidirectional and outputs realtime flowrate. Realtime pressure and temperature values may also be monitored during the draw stroke to potentially detect forms of occlusion and/or a bag empty condition based on a resulting flowrate profile. Such realtime evaluation may be used even if such measurements are not used for volume calculations.

The control unit with all fluid valves closed next causes the air pump to again vent the pump actuation chamber to atmosphere via the pneumatic valve located along the pneumatic delivery line being set to vent. Next, the control unit with all fluid valves still closed again causes the air pump to pressurize the pneumatic side of the flexible sheet to a specified pressure, e.g., five psig. At this step, air moves past the air flow sensor at varying pressures. The outputs from the air flow sensor, pressure sensor and temperature sensor located along the common line are recorded again by the control unit over multiple points in time until, again, the measured pressure reaches a specified value, e.g., five psig on the pneumatic side of the flexible sheet.

The control unit then performs a before and after calculation to approximately determine how much fresh or used PD fluid has been pulled into the pump chamber. In an embodiment, two algorithms or equations are used, wherein the first algorithm or equation is used twice, before and after each collection of flow sensor, pressure sensor and temperature sensor data. The second equation then determines the difference between the outcomes of the initial and final (before and after) uses of the first equation. The first equation used for the initial five psig application is in one embodiment:

$$\begin{matrix}\text{?} = \frac{\{ [ \sum {P}_{i} ( t ) \frac{{f}_{i} ( t ) Δ t}{{RT}_{i} ( t )} ] R \text{?} \}}{Δ \text{?}}\end{matrix} ,$$

$$\text{?} \text{indicates text missing or illegible when filed}$$

which is again derived from the Ideal Gas Law, and wherein R is a constant, f for a given point in time is the measured flowrate, P for the given point in time is the measured pressure and T for the given point in time is the measured temperature (for the initial five psig application). Vi is the initial pneumatic side air volume before drawing fresh or used PD fluid into the fluid side of the pump chamber. The first equation used for the final five psig application is then:

$$\begin{matrix}\text{?} = \frac{\{ [ \sum {P}_{f} ( t ) \frac{{f}_{f} ( t ) Δ t}{{RT}_{f} ( t )} ] R \text{?} \}}{Δ {P}_{f}}\end{matrix} ,$$

$$\text{?} \text{indicates text missing or illegible when filed}$$

wherein R is a constant, f for a given point in time is the measured flowrate, P for the given point in time is the measured pressure and T for the given point in time is the measured temperature (for the final five psig application). Vf is the final pneumatic side air volume after the fluid draw. The second equation used for determining (approximating) how much fresh or used PD fluid is drawn into the pump chamber may then be:

$$Δ V = Vf - Vi ,$$

wherein ΔV is the amount of fresh or used PD fluid that has been drawn into the pump chamber. The control unit may then use the amount drawn for a subsequent discharge stroke to know how much (approximately) fresh or used PD fluid is available to deliver to a desired destination.

The following paragraphs discuss using the methodology of the present disclosure for occlusion detection, patient or bag empty detection and air in fluid detection. For the first two (occlusion/empty) there are multiple ways for making such detections. In a first way, during a delivery stroke or suction stroke, the control unit analyzes the air flowrate profile resulting from the output of the flow sensor. The control unit detecting no change in air flowrate would determine a complete occlusion or bag/patient completely empty. The control unit detecting a lower rate of decay of air flowrate determines a partial occlusion or bag/patient near empty. In a second way, the control unit makes the determinations based on a measured suction or delivery fluid volume by movement of the flexible sheet compared against an expected suction or delivery fluid volume, wherein the expected volume is based on a volume defined by disposable pump chamber. No actual volume pumped indicates a full occlusion or full empty condition. A partial but less than expected volume pump indicates a partial occlusion or empty condition. In a third way, the control unit makes determinations based on a difference in the volume measured between a suction stroke and a delivery stroke. The third way is used in an embodiment to determine between an occlusion and an empty condition that has been detected via one of the first two ways, here attempting to push back to the same line upon which the suction stroke is made. If resistance is still found in the delivery stroke to the same line, then an occlusion is determined. If the resistance seen in the suction stroke is no longer present in the delivery stroke to the same line (here may need to pull fluid from a different source for the delivery stroke), then an empty condition is determined.

The draw approximation and subsequent accurate discharge determination may be used to detect a full or partial occlusion. The draw approximation during an occlusion would show that very little or no fresh or used PD has entered the pump chamber from the patient or from a PD fluid container or bag. The accurate discharge determination (here attempting to pump to the same line as the attempted draw) would show an inability (little or none) to push back fresh PD fluid to the patient (for a patient line occlusion) or a PD fluid container or bag (for a PD fluid line occlusion), or to push used PD fluid to a drain container or house drain. In particular, during the discharge of PD fluid, the control unit in an embodiment looks at the resulting flowrate profile to determine if a partial or complete occlusion has occurred. The control unit alternatively or additionally determines an occlusion based on a measured PD fluid discharge volume compared against an expected PD fluid discharge volume. It is contemplated for the control unit to verify a partial or complete occlusion by causing the application of different air pressures at the pneumatic side of the flexible sheet. The partial or complete occlusion should be detected at each varied air pressure as confirmation. Higher pressures may be limited by the control unit to the PD fluid supply lines and the drain line, so that the patient does not see the higher pressures along the patient line.

The draw approximation and subsequent accurate discharge determination may also be used to detect a patient empty condition or a bag empty condition. The draw approximation during a patient empty condition would show that very little used PD fluid has been pulled from the patient. The draw approximation during a container or bag empty condition would show that very little fresh PD fluid has been pulled from a PD fluid container or bag. The accurate discharge determination attempting to pump to drain (for patient empty condition) or to the patient line (for bag empty condition) would however show that a small amount of (i) effluent is able to be pushed to a house drain or drain container (for patient empty), or (ii) fresh PD fluid is able to be pushed to the patient (for bag empty).

The control unit may further be configured to detect air during the draw approximation by observing a higher than normal Vi or Vf determination. In particular, the control unit may determine the presence of air in the disposable set based on an absolute volume measurement during a draw stroke. The presence of air results in a higher Vi or Vf determination than typical (lower fluid volume), which may be due to a partial occlusion in the fluid suction line (in case of Vf) or one or more air bubble in the disposable set (for both Vi and Vf). It is contemplated for the control unit to confirm the presence of air by causing the application of different air pressures to determine Vi or Vf as applicable. Here, the presence of air causes a variation in the Vi or Vf measurement/calculation as a function of positive pressure for each of the different pressures applied, which may be observed for verification.

In light of the present disclosure, and without limiting the disclosure in any way, in a first aspect of the present disclosure, which may be combined with any other aspect, or portion thereof, a peritoneal dialysis (“PD”) system includes a disposable set including a pump chamber having a flexible sheet, a pneumatic side of the flexible sheet positioned and arranged during operation to receive pneumatic pressure; and a cycler including at least one source of positive and negative pneumatic pressure for delivering pneumatic pressure to the pump chamber, an air flow sensor, a pneumatic pressure sensor, a plurality of fluid valves, and a control unit configured to integrate outputs from the air flow sensor and the pneumatic pressure sensor over time to determine an amount of fresh or used PD fluid discharged from the pump chamber under positive pneumatic pressure and via an open one of the plurality of fluid valves.

In a second aspect of the present disclosure, which may be combined with any other aspect, or portion thereof, the at least one source of positive and negative pneumatic pressure includes an air pump.

In a third aspect of the present disclosure, which may be combined with any other aspect, or portion thereof, the PD system includes a pneumatic delivery line and a pneumatic suction line extending from the air pump, the pneumatic delivery line and the pneumatic suction line in selective pneumatic communication with the pneumatic side of the flexible sheet.

In a fourth aspect of the present disclosure, which may be combined with any other aspect, or portion thereof, the cycler includes a pump actuation chamber covered in operation by the pneumatic side of the flexible sheet, the pneumatic delivery line and the pneumatic suction line in selective pneumatic communication with the pump actuation chamber.

In a fifth aspect of the present disclosure, which may be combined with any other aspect, or portion thereof, the PD system includes a pneumatic rigid plastic shell connected to the pump chamber so as to seal the flexible sheet between the pneumatic rigid plastic shell and the pump chamber, the pneumatic delivery line and the pneumatic suction line in selective pneumatic communication with a port provided by the pneumatic rigid plastic shell.

In a sixth aspect of the present disclosure, which may be combined with any other aspect, or portion thereof, the pneumatic delivery line and the pneumatic suction line extend to a common pneumatic line, the air flow sensor and the pneumatic pressure sensor in operable communication with the common pneumatic line.

In a seventh aspect of the present disclosure, which may be combined with any other aspect, or portion thereof, the PD system further includes an air temperature sensor in operable communication with the common pneumatic line, the control unit further configured to integrate outputs from the air temperature sensor to determine the amount of fresh or used PD fluid that has been discharged from the pump chamber.

In an eighth aspect of the present disclosure, which may be combined with any other aspect, or portion thereof, the PD system includes a first pneumatic valve located along the delivery line and a second pneumatic valve located along the pneumatic suction line, the first and second pneumatic valves providing selective pneumatic communication with the pneumatic side of the flexible sheet.

In a ninth aspect of the present disclosure, which may be combined with any other aspect, or portion thereof, the at least one source of positive and negative pneumatic pressure includes positive and negative pneumatic storage vessels.

In a tenth aspect of the present disclosure, which may be combined with any other aspect, or portion thereof, the control unit is configured to integrate outputs from the air flow sensor and the pneumatic pressure sensor over time using an algorithm derived from the Ideal Gas Law.

In an eleventh aspect of the present disclosure, which may be combined with any other aspect, or portion thereof, the control unit is configured to integrate outputs from the air flow sensor and the pneumatic pressure sensor over time for (i) an initial pneumatic pressurization of the pump chamber to determine an initial chamber volume and (ii) a final pneumatic pressurization of the pump chamber to determine a final chamber volume, the control unit further configured to determine an amount of fresh or used PD fluid drawn into the pump chamber by subtracting the initial chamber volume from the final chamber volume.

In a twelfth aspect of the present disclosure, which may be combined with any other aspect, or portion thereof, the control unit is configured to cause negative pneumatic pressure to be applied to the pump chamber with one of the plurality of fluid valves opened between the initial pneumatic pressurization and the final pneumatic pressurization.

In a thirteenth aspect of the present disclosure, which may be combined with any other aspect, or portion thereof, the control unit is configured to cause at least one pneumatic line to be vented prior to each of the initial pneumatic pressurization and the final pneumatic pressurization.

In a fourteenth aspect of the present disclosure, which may be combined with any other aspect, or portion thereof, the control unit is further configured to use (a) the output from the air flow sensor or (b) the amount of fresh or used PD fluid drawn into the pump chamber to at least partially determine at least one of (i) a partial or full line occlusion, (ii) a PD fluid container or patient empty condition, or (iii) a presence of air.

In a fifteenth aspect of the present disclosure, which may be combined with any other aspect, or portion thereof, the control unit is configured to use the amount of fresh or used PD fluid discharged from the pump chamber to confirm or fully determine at least one of (i) the partial or full line occlusion, or (ii) the PD fluid container or patient empty condition.

In a sixteenth aspect of the present disclosure, which may be combined with any other aspect, or portion thereof, the control unit is configured to attempt to have fresh or used PD fluid pushed through a same line from which fresh or used PD fluid has just previously been subjected to a fresh or used PD fluid draw to determine between (i) the partial or full line occlusion, and (ii) the PD fluid container or patient empty condition.

In a seventeenth aspect of the present disclosure, which may be combined with any other aspect, or portion thereof, the PD system further includes an air temperature sensor, and wherein the control unit is configured to determine the amount of fresh or used PD fluid discharged from the pump chamber according to the equation:

$$Δ V = \frac{[ \sum P ( t ) \frac{f ( t ) Δ t}{RT ( t )} ]}{P} RT ,$$

wherein R is a constant, f is measured air flowrate, P is measured air pressure and T is measured temperature.

In an eighteenth aspect of the present disclosure, which may be combined with any other aspect, or portion thereof, a peritoneal dialysis (“PD”) system including a disposable set including a pump chamber having a flexible sheet, a pneumatic side of the flexible sheet positioned and arranged during operation to receive pneumatic pressure; and a cycler including at least one source of positive and negative pneumatic pressure for delivering pneumatic pressure to the pump chamber, an air flow sensor, a pneumatic pressure sensor, a plurality of fluid valves, and a control unit configured to integrate outputs from the air flow sensor and the pneumatic pressure sensor over time for (i) an initial pneumatic pressurization of the pump chamber to determine an initial chamber volume and (ii) a final pneumatic pressurization of the pump chamber to determine a final chamber volume, the control unit further configured to determine an amount of fresh or used PD fluid drawn into the pump chamber by subtracting the initial chamber volume from the final chamber volume.

In a nineteenth aspect of the present disclosure, which may be combined with any other aspect, or portion thereof, the control unit is configured to cause negative pneumatic pressure to be applied to the pump chamber with one of the plurality of fluid valves opened between the initial pneumatic pressurization and the final pneumatic pressurization.

In a twentieth aspect of the present disclosure, which may be combined with any other aspect, or portion thereof, the control unit is configured to cause at least one pneumatic line to be vented prior to each of the initial pneumatic pressurization and the final pneumatic pressurization.

In a twenty-first aspect of the present disclosure, which may be combined with any other aspect, or portion thereof, the control unit is further configured to use (a) the output from the air flow sensor or (b) the amount of fresh or used PD fluid drawn into the pump chamber to at least partially determine at least one of (i) a partial or full line occlusion, (ii) a PD fluid container or patient empty condition, or (iii) a presence of air.

In a twenty-second aspect of the present disclosure, which may be combined with any other aspect, or portion thereof, the control unit is configured to attempt to have fresh or used PD fluid pushed through a same line from which fresh or used PD fluid has just previously been subjected to a fresh or used PD fluid draw to determine between (i) the partial or full line occlusion, and (ii) the PD fluid container or patient empty condition.

In a twenty-third aspect of the present disclosure, which may be combined with any other aspect, or portion thereof, the PD system further includes an air temperature sensor, and wherein the control unit is configured to integrate outputs from the air flow sensor, the pneumatic pressure sensor and the air temperature sensor over time for determining the an amount of fresh or used PD fluid drawn into the pump chamber according to the equations:

$$Δ V = Vf - {V}_{i} , \qquad ( i )$$

$$\begin{matrix}\text{?} = \frac{\{ [ \sum {P}_{f} ( t ) \frac{{f}_{f} ( t ) Δ t}{{RT}_{f} ( t )} ] R \text{?} \}}{Δ {P}_{f}}\end{matrix} , \qquad ( ii )$$

$$and$$

$$\begin{matrix}{V}_{i} = \frac{\{ [ \sum \text{?} ( t ) \frac{{f}_{i} ( t ) Δ t}{{RT}_{i} ( t )} ] R \text{?} \}}{Δ {P}_{i}}\end{matrix} , \qquad ( iii )$$

$$\text{?} \text{indicates text missing or illegible when filed}$$

wherein R is a constant, f is measured air flowrate, P is measured air pressure and T is measured temperature.

In a twenty-fourth aspect, which may be combined with any other aspect, or portion thereof, any of the features, functionality and alternatives described in connection with any one or more of FIGS. 1 to 7 may be combined with any of the features, functionality and alternatives described in connection with any other of FIGS. 1 to 7.

In light of the above aspects and the disclosure herein, it is an advantage of the present disclosure to provide a relatively volumetrically accurate automated peritoneal dialysis (“APD”) cycler.

It is another advantage of the present disclosure to provide an APD cycler that achieves relatively precise pressure control.

It is a further advantage of the present disclosure to provide a relatively quiet APD cycler.

It is still another advantage of the present disclosure to provide an APD cycler operated using the Ideal Gas Law, which does not require a reference chamber.

It is still a further advantage of the present disclosure to provide an APD system that is able to build motive fluid or pneumatic pressure in a relatively simple manner, reducing part count and complexity.

It is yet another advantage of the present disclosure to provide an APD system that employs a relatively low cost disposable set.

It is yet a further advantage of the present disclosure to provide an APD system operated using the Ideal Gas Law, which may operate without positive and negative pneumatic reservoirs.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic elevation view of one embodiment of a pneumatically driven automated peritoneal dialysis (“APD”) cycler of the present disclosure.

FIG. 2 is a process flow diagram illustrating one embodiment for method of accurately determining an amount of fresh or used PD fluid discharged pneumatically from a pump chamber.

FIG. 3 is a graph illustrating air flowrate, pneumatic pressure and fluid flowrate variation over time using the method of FIG. 2.

FIG. 4 is a process flow diagram illustrating one embodiment for method of approximating an amount of fresh or used PD fluid drawn pneumatically into a pump chamber.

FIGS. 5 to 7 are graphs illustrating air flowrate and pneumatic pressure variation over time using the method of FIG. 4.

### DETAILED DESCRIPTION

### System Overview

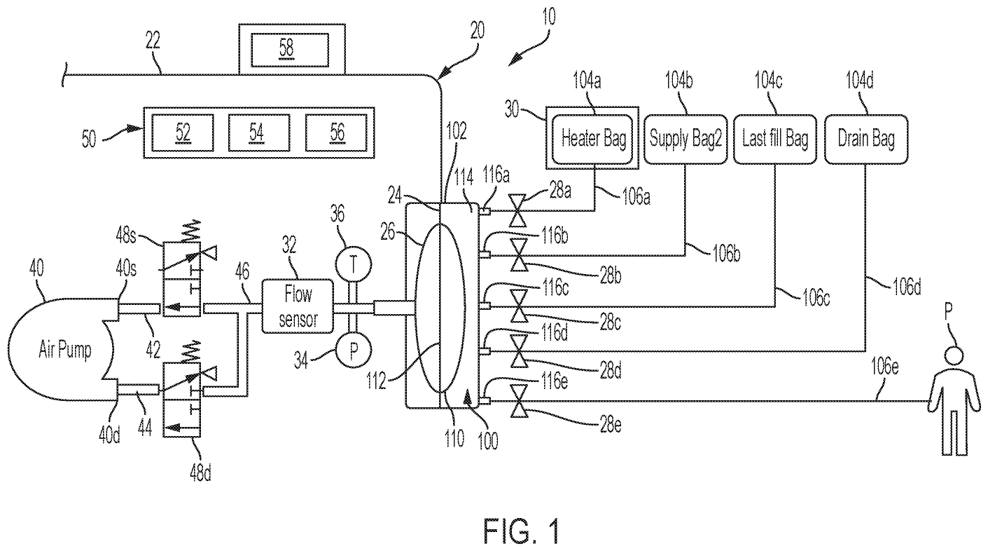

Referring now to the drawings and in particular to FIG. 1, an embodiment of system 10 includes an automated peritoneal dialysis (“APD”) cycler 20 having a housing 22, which operates with a disposable set 100 that organizes tubing and performs many functions discussed herein. Disposable set 100 includes cassette 102 having or defining a pump chamber 110, which in one embodiment is covered on at least one side by a flexible plastic sheet, diaphragm or membrane 112.

As illustrated in FIG. 1, disposable set 100 including pump chamber 110 is inserted for operation inside of APD cycler 20, for example, in between an actuation surface 24 and a door (not illustrated) of the APD cycler. The door may hinge open via one or more hinge (not illustrated) provided by cycler housing 22, adjacent to actuation surface 24, which includes or defines a pump actuation chamber 26. In the illustrated implementation, pump chamber 110 of disposable set 100 is mounted so that flexible plastic sheet 112 of pump chamber 110 when mounted for operation is located facing actuation surface 24 of cycler 20 so as to overlie pump actuation chamber 26. The door when closed constrains pump chamber 110 and flexible sheet 112 against pump actuation chamber 26 during operation in one embodiment.

In one possible configuration for system 10 as illustrated in FIG. 1, disposable set 100 includes multiple PD fluid containers or bags 104a to 104c, wherein one of the containers, e.g., container 104a, is placed on top of housing 22, which includes a dialysis fluid heater 30 to heat PD fluid located originally in container 104a as well as dialysis fluid pumped to container 104a from second and third PD fluid containers 104b and 104c for subsequent patient fills. In an alternative embodiment, plate or batch heater 30 is replaced with an inline dialysis fluid heater (not illustrated), which heats fresh dialysis fluid as it flows through a patient line to the patient. Here, a serpentine inline heating pathway may be provided along the patient line to aid the inline heating. The serpentine inline heating pathway may be placed on top of cycler 20 or along actuation surface 24 for heating, e.g., via resistive plate heating.

Disposable pump chamber 110 is oriented vertically in the illustrated embodiment of FIG. 1, wherein fluid tubes or lines run horizontally from pump chamber 110. In the illustrated embodiment, pump chamber 110 includes a fluid contacting rigid plastic shell 114 of cassette 102, to which flexible sheet 112 is attached, e.g., ultrasonically sealed. FIG. 1 illustrates that cassette 102 includes or defines a first dialysis fluid port 116a that accepts a first PD fluid line 106a in a sealed manner, wherein first PD fluid line 106a extends to fresh PD fluid container 104a located on top of housing 22 in one embodiment, wherein container 104a may double as the fluid heating container. Disposable cassette 102 includes or defines a second dialysis fluid port 116b that accepts a second PD fluid line 106b in a sealed manner, wherein second PD fluid line 106b extends to fresh dialysis fluid container 104b. Disposable cassette 102 includes or defines a third dialysis fluid port 116c that accepts a third PD fluid line 106c in a sealed manner, wherein third PD fluid line 106c extends to fresh dialysis fluid container 104c. Disposable cassette 102 includes or defines a drain port 116d that accepts a drain line 106d in a sealed manner, wherein drain line 106d extends to a drain container 104d or house drain, e.g., toilet, bathtub or sink (not illustrated). Disposable cassette 102 further includes or defines a patient port 116e that accepts a patient line 106e in a sealed manner, wherein patient line 106e extends to a patient connector (not illustrated) that connects to a patient's transfer set and communicates fluidly with the indwelling peritoneal dialysis catheter of patient P.

Disposable set 100 may alternatively provide more or less than the illustrated five fluid lines, e.g., the number of fresh dialysis fluid containers may be varied. Fresh PD fluid containers 104a to 104c may contain the same or different types and volumes of fresh PD fluids. For example, fresh PD fluid containers 104a to 104c may contain different levels of dextrose or glucose. One of the containers may contain a different formulation of fresh PD fluid, e.g., icodextrin, for a patient's last fill.

To load disposable set 100 and pump chamber 110 of disposable cassette 102 for operation, patient P or a caregiver in one embodiment opens the cycler door and loads disposable set 100 into the inside of the door and against actuation surface 24. Patient P or the caregiver then closes the door, causing (i) pump chamber 110 to come into registry with pump actuation chamber 26 and (ii) fluid lines 106a to 106e to come into registry, respectively, with fluid valves, such as pinch valves 28a to 28e located along actuation surface 24 for example. The patient or caregiver then loads fresh PD fluid container 104a onto resistive plate or batch dialysis fluid heater 30 assuming that batch heating is provided. Additional dialysis fluid containers or bags 104b and 104c may be located in a convenient position on the table, nightstand, desk, etc.

Pinch valves 28a to 28e may each be an electrically actuated solenoid valve. Pinch valves 28a to 28e are fail safe in one embodiment, such that upon a power loss the valves are biased to close their respective fluid lines 106a to 106e. It should be appreciated that fluid valves 28a to 28e may alternatively be pneumatic valves, such as pillow valves, that likewise actuate respective flexible fluid lines 106a to 106e. Fluid valves 28a to 28e may further alternatively be pneumatic valves that actuate dedicated areas of flexible sheet 112 of disposable cassette 102.

It should be appreciated that pump chamber 110 may include an air contacting rigid plastic shell (not illustrated) in addition to fluid contacting fluid contacting rigid plastic shell 114, wherein the pair of shells surround flexible sheet 112. Here, air contacting rigid plastic shell (not illustrated) includes or defines a rigid plastic pneumatic port that interfaces in a releasably sealed manner with a pneumatic actuation aperture (not illustrated) located along actuation surface 24, wherein the pneumatic actuation aperture replaces pump actuation chamber 26. In any embodiment for pump chamber 110, flexible sheet 112 is (i) pulled under negative pressure to pull fresh or used PD fluid into disposable pump chamber 110, and (ii) pushed under positive pressure to push fresh or used PD fluid from disposable pump chamber 110.

Rigid plastic shell 114 and any other part of disposable cassette 102, flexible sheet, fluid lines 106a to 106e, and PD fluid containers or bags 104a to 104d of disposable set 100 may be made of one or more plastic, e.g., polyvinylchloride (“PVC”) or a non-PVC material, such as polyethylene (“PE”), polyurethane (“PU”) or polycarbonate (“PC”). Housing 22 of cycler 20 may be made of any of the above plastics, and/or of metal, e.g., stainless steel, steel and/or aluminum.

FIG. 1 further illustrates that cycler 20 provides an air pump 40 located within housing 22 for pneumatically driving disposable pump chamber 110. Air pump 40 may be single direction or bidirectional and can supply positive and negative pneumatic pressure, e.g., positive pneumatic pressure out of delivery port 40d and negative pneumatic pressure from suction port 40s. Suitable air pumps 40 may for example be diaphragm pumps, scroll pumps or piston pumps. Air pump 40 may be electrically actuated such that the amount of current delivered to the air pump determines the speed of the pump and the pressure of air delivered to pump chamber 110.

Air pump 40 may or may not operate with pneumatic storage vessels that store positive and negative pneumatic pressure. The storage vessels if provided are used to deliver positive and negative pneumatic pressure to pump chamber 100. If the storage vessels are not provided in the case of FIG. 1, air pump 40 is configured to provide both positive and negative pressure air to disposable pump chamber 110 via pneumatic lines. The pneumatic lines in the illustrated embodiment include a pneumatic suction line 42 extending from suction port 40s and a pneumatic delivery line 44 extending from delivery port 40d. Pneumatic suction line 42 includes or is provided with a pneumatic valve 48s that either opens or closes pneumatic line 42 to atmosphere. Pneumatic delivery line 44 includes or is provided with a pneumatic valve 48d that either opens or closes pneumatic line 44 to atmosphere. Pneumatic suction line 42 and pneumatic delivery line 44 meet at a common pneumatic line 46 that extends to reusable pump actuation chamber 26 of cycler 20.

To apply negative pneumatic pressure to pump actuation chamber 26, pneumatic valve 48d located along pneumatic delivery line 44 is set to vent, while pneumatic valve 48s located along pneumatic suction line 42 is closed to atmosphere. Air pump 40 is actuated to create a set negative pneumatic pressure in pneumatic suction line 42 and common pneumatic line 46, which pulls on flexible pumping sheet 112 of the pump chamber 110, which is coupled against pump actuation chamber 26. Air pulled to create the negative pneumatic pressure is vented to atmosphere via pneumatic valve 48d located along pneumatic delivery line 44.

To apply positive pneumatic pressure to pump actuation chamber 26, pneumatic valve 48s located along pneumatic suction line 42 is set to vent, while pneumatic valve 48d located along pneumatic delivery line 44 is closed to atmosphere. Air pump 40 is actuated (e.g., in the same direction as above) to create a set positive pneumatic pressure in pneumatic delivery line 44 and common pneumatic line 46, which pushes on flexible pumping sheet 112 of disposable pump chamber 110, which is again coupled against pump actuation chamber 26. Air used to create positive pneumatic pressure is pulled in via pneumatic valve 48s located along pneumatic suction line 42.

In an embodiment, multiple sensors are located along common pneumatic line 46, including an air flow sensor 32, a pneumatic pressure sensor 34 and a pneumatic temperature sensor 36. The outputs from each of the air flow, pressure and temperature sensors are used for the Ideal Gas Law fluid volume determinations discussed herein. The output from pressure sensor 34 is also used as feedback for the control of air pump 40 to deliver PD fluid to and from patient P at desired positive and negative pressures, respectively, as described herein.

FIG. 1 further illustrates that cycler 20 includes a control unit 50 having one or more processor 52, one or more memory 54 and a video controller 56 operating with a user interface 58 provided to control each of fluid valves 28a to 28e, air pump 40, each pneumatic valve 48d, 48s, and PD fluid heater 30 and to receive signals from each of air flow sensor 32, pneumatic pressure sensor 34 and pneumatic temperature sensor 36. Additional sensors outputting to control unit 50 may include one or more additional temperature sensor (not illustrated) operable with PD fluid heater 30, a priming sensor (not illustrated, e.g., ultrasonic, optical, capacitive or inductive), and/or one or more conductivity sensor. Control unit 50 may use feedback from the one or more temperature sensor operable with PD fluid heater 30 to adjust the amount of power delivered to the heater, e.g., via a proportional, integral, derivative (“PID”) routine, so that heated, fresh PD fluid is delivered to patient P at a desired temperature, e.g., 37° C.

User interface 58 may be provided with a touchscreen and/or electromechanical pushbuttons to allow the user or patient P to enter parameters for treatment and a display screen for providing information, such as treatment status information. Control unit 50 may also include a transceiver (not illustrated) and a wired or wireless connection to a network, e.g., the internet, for sending treatment data to and receiving prescription instructions from a doctor's or clinician's server interfacing with a doctor's or clinician's computer.

To prime first PD fluid/heater bag line 106a, control unit 50 causes first fluid valve 28a to open and air pump 40 to place flexible sheet 112 under negative pressure to pull fresh PD fluid from first PD fluid container or bag 104a into disposable pump chamber 110. If the volume of disposable pump chamber 110 is greater than that of first PD fluid/heater bag line 106a, then line 106a may be fully primed after one draw stroke, wherein control unit 50 may cause first fluid valve 28a to close, drain valve 28d to open and air pump 40 to place flexible sheet 112 under positive pressure to push fresh PD from pump chamber 110, down drain line 106d. The above sequence may be repeated one or more time to ensure that first PD fluid/heater bag line 106a is fully primed.

If the volume of disposable pump chamber 110 is less than that of first PD fluid/heater bag line 106a, then control unit 50 may cause first fluid valve 28a to close, drain valve 28d to open and air pump 40 to place flexible sheet 112 under positive pressure to push air pulled into pump chamber 110, down drain line 106d. The above sequence is then repeated one or more time, each time pushing air down drain line 106d, until first PD fluid/heater bag line 106a is fully primed.

Control unit 50 primes second and third PD fluid lines 106b and 106c in the same way as described above (including all alternatives) but instead using fluid valves 28b and 28c, respectively.

Control unit 50 primes patient line 106e by causing first fluid valve 28a to open and air pump 40 to place flexible sheet 112 under negative pressure to pull fresh PD fluid from first PD fluid/heater bag line 106a, which has been fully primed, into disposable pump chamber 110. Control unit 50 then causes first fluid valve 28a to close, patient valve 28e to open and air pump 40 to place flexible sheet 112 under positive pressure to push fresh PD fluid from pump chamber 110 down patient line 106e. Control unit 50 repeats the above sequence as many times as needed to fully prime patient line 106e. It is contemplated that housing 22 of cycler 20 provide a prime sensor/bubble detector (not illustrated), e.g., an ultrasonic, optical capacitive or inductive sensor, which grasps and operates with a distal end of patient line 106e to look for fresh PD fluid during priming to know that patient line 106e has been fully primed.

Positive and negative pumping pressures for any of the priming sequences above may be higher than those used for pumping to and from patient P to reduce setup and treatment time. The limits for pumping pressures used during the priming sequences may be set by one or more of the capabilities of air pump 40 and/or structural limitations imposed by flexible sheet 112.

The first sequence after priming disposable set 100 in the manner described above is often to initially drain patient P who is full from a previous treatment or midday exchange. To drain patient P, control unit 50 causes patient valve 28e to open and air pump 40 to place flexible sheet 112 under negative pressure to pull used PD fluid from patient P, through patient line 106e, into pump chamber 110. When pump chamber 110 is full of used PD fluid, control unit 50 causes patient valve 28e to close, drain valve 28d to open, and air pump 40 to place flexible sheet 112 under positive pressure to push used PD fluid from pump chamber 110, down drain line 106d. Control unit 50 repeats the above sequence as many times as needed to fully drain patient P. Structure and methodology for knowing how much used PD fluid is removed from patient P is discussed herein, which also shows that patient draining pressure is controlled and set to a safe level.

The sequence performed after draining patient P in the manner described above is to then fill patient P with fresh PD fluid. To fill patient P, control unit 50 causes first fluid valve 28a to open and air pump 40 to place flexible sheet 112 under negative pressure to pull fresh, heated PD fluid from first PD fluid/heating container 104a, through first PD fluid line 106a, into pump chamber 110. When pump chamber 110 is full of fresh, heated PD fluid, control unit 50 causes first fluid valve 28a to close, patient valve 28e to open, and air pump 40 to place flexible sheet 112 under positive pressure to push fresh, heated PD fluid from pump chamber 110, through patient line 106e, to patient P. Control unit 50 repeats the above sequence as many times as needed to fully fill patient P. Structure and methodology for knowing how much fresh, heated PD fluid is delivered to patient P is discussed herein, which also shows that patient filling pressure is controlled and set to a safe level.

The sequence after filling patient P in the manner described above is to allow the fresh PD fluid to dwell within the patient for a specified period of time, e.g., perhaps two hours. During this time, it is contemplated for control unit 50 of system 10 to replenish first PD fluid/heating container 104a with fresh PD fluid, if needed, from second and third containers 104b and 104c for subsequent patient fills. To replenish first PD fluid/heating container 104a, control unit 50 causes second fluid valve 28b or third fluid valve 28c to open and air pump 40 to place flexible sheet 112 under negative pressure to pull fresh PD fluid from second PD fluid container 104b or third PD fluid container 104b, through second PD fluid line 106b or third PD fluid line 106b, into pump chamber 110. When pump chamber 110 is full of fresh PD fluid, control unit 50 causes second fluid valve 28b or third fluid valve 28c to close, first fluid valve 28a to open, and air pump 40 to place flexible sheet 112 under positive pressure to push fresh PD fluid from pump chamber 110, through first PD fluid line 106a, to first PD fluid/heating container 104a. Control unit 50 repeats the above sequence as many times as needed to fill first PD fluid/heating container 104a with a needed amount of fresh PD fluid for the next patient fill. The structure and methodology for knowing how much fresh PD fluid is pumped may be used here to know how much fresh PD fluid is transferred to first PD fluid/heating container 104a. Higher system pressures may be used here to reduce replenishing time because patient P is not involved with the transfer. Control unit 50 may also power PD fluid heater 30 as soon as enough fresh PD fluid is replenished into container 104a to support safe fluid heating.

### Volumetric Accuracy

The structure and functionality for knowing how much fresh or used PD fluid is transferred via pump chamber 110 is now discussed. In one embodiment, control unit 50 is programmed to cause (i) approximate PD fluid volume measurements to be made for anytime that fresh or used PD fluid is drawn into pump chamber 110 (is under negative pneumatic pressure) and (ii) accurate PD fluid volume measurements to be made for anytime that fresh or used PD fluid is discharged from pump chamber 110 (is under positive pneumatic pressure). The approximate PD fluid volume measurements made for drawing PD fluid are accurate enough to determine if an occlusion exists in a line or if a currently used PD fluid container or bag 104a to 104c is at or near empty. The accurate PD fluid volume measurements made for discharging PD fluid are accurate enough to ensure (i) that prescribed patient fill volumes are met and (ii) that patient drain volumes are accurate so that an accurate determination of the amount of ultrafiltration (“UF”) removed from patient P may be determined. The accurate PD fluid volume measurements made for discharging PD fluid are also accurate enough to determine line occlusions and to confirm container or bag empty conditions.

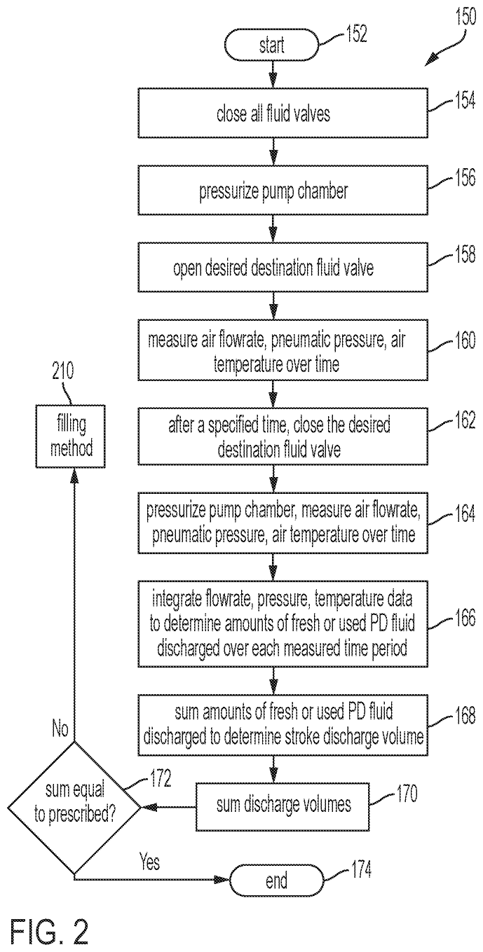

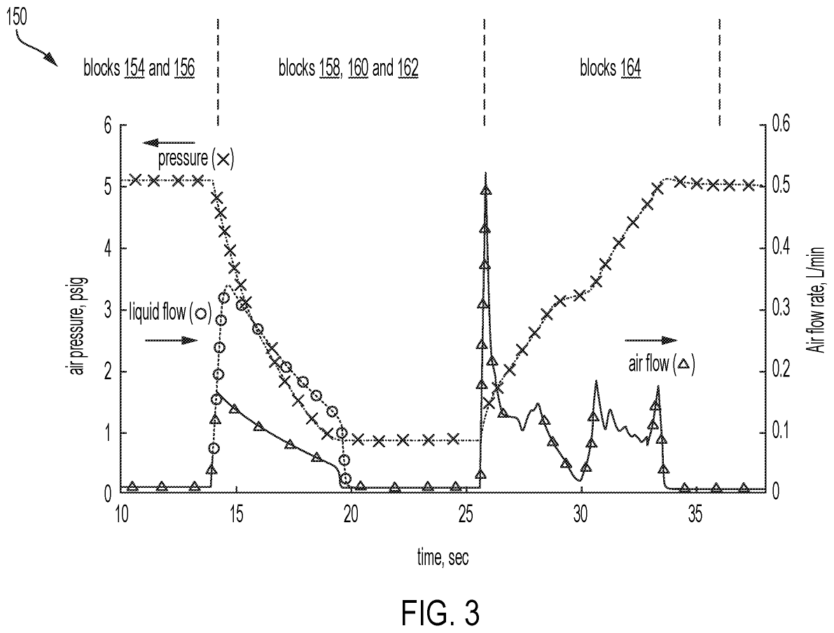

Referring now to FIGS. 2 and 3, method 150 begins at oval 152 and illustrates that control unit 50 performs the accurate (discharging) PD fluid volume measurements by closing all fluid valves 28a to 28e at block 154 and causing air pump 40 to pressurize the pneumatic side of flexible sheet 112 to a specified pressure, e.g., five psig, at block 156. Blocks 154 and 156 may occur simultaneously, or in one preferred embodiment block 154 occurs before block 156 so that there is no membrane movement or fluid delivery and an initial pressure is established, which is then matched at block 164.

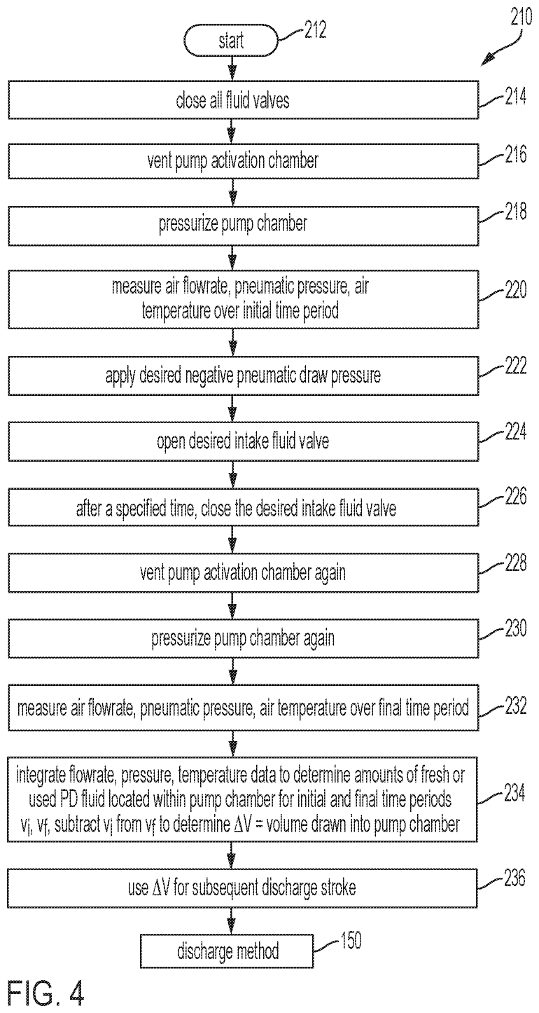

Method 150 illustrates an example method for discharging fresh or used PD fluid, while method 210 of FIG. 4 illustrates an example method for filling or drawing fresh or used PD fluid into pump chamber 110. The methods are accordingly implemented in system 10 sequentially, discharging then filling or filling then discharging. There is accordingly overlap between the two methods, where one or more step of method 150 may have already been performed in a previous implementation of method 210, or vice versa. One example is that the pressurizing of pump actuation chamber 26 at block 156 of discharge method 150 may have already taken place at the pressurizing of pump actuation chamber 26 at block 230 of filling method 210. So in the graph of FIG. 3, blocks 154 and 156 are illustrated, however, pressurization to the commanded pressure of five psig has already taken place at block 230 of the previous implementation of filling method 210. If for some reason the pressure at time 10 seconds in FIG. 3 is less than the commanded pressure of five psig, then FIG. 3 would show an additional pressurization to the commanded pressure, which occurs at blocks 154 and 156. Blocks 154 and 156 are accordingly illustrated in FIG. 3 even though they might not be needed.

At block 158, control unit 50 causes a desired discharge fluid valve 28a (replenishing), 28d (patient draining), or 28e (patient filling) to open, allowing the five psig pneumatic pressure to move flexible sheet 112, in turn causing fresh or used PD fluid to be discharged from pump chamber 110. At this time, air also moves past the air flow sensor 32 at varying pressures as measured by pressure sensor 34. In an optional embodiment, during the discharge of PD fluid from pump chamber 110, control unit 50 causes air pump 40, under feedback control from pressure sensor 34, to pressurize the pneumatic side of the flexible sheet 112 at pump actuation chamber 26 to a specified pressure, e.g., at or just below five psig, which reduces or virtually eliminates repressurization time after the discharge of fresh or used PD fluid has been completed (at block 164). At block 160, the outputs from air flow sensor 32, pressure sensor 34 and temperature sensor 36 located along common pneumatic line 46 are recorded by control unit 50 over multiple, e.g., many, points in time while the desired discharge fluid valve 28a, 28d, 28d is open. Blocks 158 and 160 may occur simultaneously. At block 162, after a set period of time, control unit 50 causes the desired discharge fluid valve 28a, 28d, 28d to close.

The graph of FIG. 3 illustrates for blocks 158 to 162 that when desired discharge fluid valve 28a, 28d, 28e is opened at about 14 seconds, the pressure begins to fall. Air flow spikes and then falls as the pressure dissipates. Fresh or used PD fluid flow also spikes and then falls as the pressure dissipates. When the desired discharge fluid valve 28a, 28d, 28e is closed at about 20 seconds, the pressure flattens at just below one psig, while air and fresh or used PD fluid flow stops.

At block 164, control unit 50, if needed, causes air pump 40 to repressurize the pneumatic side of flexible sheet 112 to a specified pressure, e.g., five psig. As discussed above, via the use of pressure feedback, the repressurization at block 164 may be fully or virtually eliminated. The repressurization at block 164 is provided if needed, however, so that the pneumatic side of flexible sheet 112 at pump actuation chamber 26 is pressurized back to the initial delivery pressure. Matching the initial delivery pressure enables the ΔV equations discussed next to be independent of the initial number of moles of air in pump actuation chamber 26, along the pneumatic side of flexible sheet 112. FIG. 3 illustrates the repressurization beginning at about 25 seconds. Here, during block 164, pneumatic pressure builds to the set pressure, e.g., five psig, while the air flow spikes and then fluctuates up and down, eventually falling to zero.

At block 166, at the end of each of time period in which air flow sensor 32, pressure sensor 34 and temperature sensor 36 data is being continuously monitored and recorded, control unit 50 feeds the data into an algorithm that determines the amount of fresh or used PD fluid discharged from pump chamber 110 over that time period. The algorithm is in one embodiment:

$$Δ V = \frac{[ \sum P ( t ) \frac{f ( t ) Δ t}{RT ( t )} ]}{P} RT ,$$

which is derived from

$$Δ V = \frac{\sum dn}{P} RT ,$$

which is derived from the Ideal Gas Law, and wherein R is a constant, f for a given point in time is the measured flowrate, P for the given point in time is the measured pressure and T for the given point in time is the measured temperature. In particular,

∫fdt≅f(t)Δt,
such that at each sampled data point, Δt=1/sampling rate, Hz, and f, P, T are measured values at that sampled data point time.

Block 168 is an optional block. If the discharge stroke described up to block 166 is not a full discharge of fresh or used PD fluid from pump chamber 110, then blocks 154 to 166 are repeated until pump chamber 110 is fully discharged. If multiple discharge strokes are needed, control unit 50 sums the amounts of fresh or used PD fluid discharged from pump chamber 110 over each discharge stoke to determine a stroke discharge volume. If instead, pump chamber 110 is repeatedly fully discharged over a single discharge stroke, then block 168 is not needed.

At block 170, control unit 50 then sums each of the stroke discharge volumes to determine a fresh PD fluid fill volume delivered to the patient, a used PD fluid drain volume removed from the patient, or a fresh fluid replenishing amount delivered to fresh PD fluid container 104a. At diamond 172, control unit 50 determines if the summed stroke discharge volumes meet or exceed a prescribed discharge volume? If not, then discharge method 150 moves to filling method 210 as illustrated in FIG. 2, so that pump chamber 110 may receive a new volume of fresh or used PD fluid. If so, control unit 50 is programmed to stop incrementing a fresh PD fluid fill volume when it meets or slightly exceeds a prescribed PD fluid fill volume. Control unit 50 is programmed to stop incrementing a used PD fluid drain volume when it meets or exceeds a prescribed PD fluid drain volume or when a flowrate of used PD fluid drops to a point indicating that the patient is effectively empty. Control unit 50 is programmed to stop incrementing a fresh PD fluid replenishing volume (delivering fresh PD fluid to fluid heating container 104a) when it meets or exceeds the next prescribed PD fluid fill volume. In any of the above scenarios, at oval 174, method 150 ends.

Referring now to FIGS. 4 to 7, filling method 210 begins at oval 212 and illustrates that control unit 50 begins the approximate PD fluid volume measurements for drawing fresh or used PD fluid into pump chamber 110 by closing all fluid valves 28a to 28e at block 214 and at block 216 causing air pump 40 to vent pump actuation chamber 26 to atmosphere via pneumatic valve 48d located along pneumatic delivery line 44 being set to vent. At block 218, control unit 50 with all fluid valves 28a to 28e still closed causes air pump 40 to pressurize the pneumatic side of flexible sheet 112 to a specified pressure, e.g., five psig, causing air to move past air flow sensor 32 at varying pressures as measured by pressure sensor 34. At block 220, during the pressurization, the outputs from air flow sensor 32, pressure sensor 34 and temperature sensor 36 located along common pneumatic line 46 are recorded by control unit 50 over multiple points in time until the pressure measured at pressure sensor 34 reaches a commanded value, e.g., five psig, on the pneumatic side of flexible sheet 112 at pump actuation chamber 26. Since fluid side valves 28a to 28e are closed, fluid side pressure in pump chamber 110 follows the air side pressure in pump actuation chamber 26 over time.

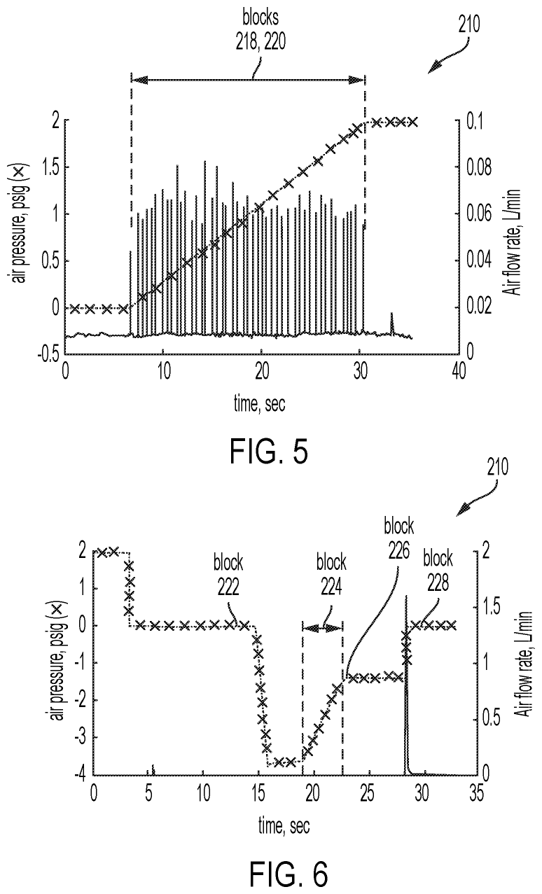

FIG. 5 does not illustrate the venting of pump actuation chamber 26 at blocks 214 and 216. FIG. 5 does however illustrate that the operation of blocks 218 and 220 occurs over a period from about seven seconds to about thirty seconds. Over this time period, the pressurization begins at zero and ends at about two psig. During the same time period, the air flow fluctuates up and down, which is recorded by air flow sensor 32 along with the pneumatic pressure recorded by pressure sensor 34. At about thirty seconds, the pneumatic pressure flattens out at about two psig and the air flow drops to zero.

At block 222, control unit 50 causes air pump 40 to apply a desired draw pressure, e.g., negative three psig pneumatic pressure, to attempt to pull flexible sheet 112. At block 224, control unit 50 causes a desired intake fluid valve 28a to 28c or 28e to open, in turn causing fresh or used PD fluid to be drawn into pump chamber 110 under the negative pressure applied at block 222. It should be appreciated that either block 222 or 224 may begin before the other or occur simultaneously. Control unit 50 does not take realtime flowrate, pressure and temperature measurements during the actual fresh or used PD fluid draw as part of the approximation. The control unit instead performs a before and after evaluation as shown below. In an optional embodiment, air flow sensor 32 is bidirectional and outputs realtime flowrate to control unit 50. Realtime pressure and temperature values may also be monitored during the draw stroke by control unit 50 to potentially detect forms of occlusion and/or a bag empty condition based on a resulting flowrate profile. Such realtime evaluation may be used even if such measurements are not used by control unit 50 for volume calculations.

At block 226, after a specified amount of time, which may be an amount of time expected to fully fill the pump chamber, control unit 50 causes intake fluid valve 28a to 28c or 28e to close. At block 228, control unit 50 with all fluid valves closed causes air pump 40 to again vent pump actuation chamber 26 to atmosphere via pneumatic valve 48d located along pneumatic delivery line 44 being set to vent.

FIG. 6 illustrates that at about sixteen seconds, a negative pneumatic pressure of negative three to four psig is applied at block 222. At about nineteen seconds, desired intake fluid valve 28a to 28c or 28e is opened at block 224, causing the applied negative pneumatic pressure to dissipate to about −1.5 psig, while fresh or used PD fluid is pulled into pump chamber 110. At about twenty-three seconds, desired intake fluid valve 28a to 28c or 28e is closed at block 226, causing the negative pressure to flatten. At about twenty-eight seconds, pneumatic valve 48d located along pneumatic delivery line 44 being set to vent at block 228, so that the negative pneumatic pressure dissipates to zero.

At block 230, control unit 50 with all fluid valves 28a to 28e still closed again causes air pump 40 to pressurize the pneumatic side of flexible sheet 112 (pump actuation chamber 26) to a specified pressure, e.g., five psig. Here, air moves past air flow sensor 32 at varying pressures as measured by pressure sensor 34. At block 232 (which may occur at the same time as block 230) the outputs from air flow sensor 32, pressure sensor 34 and temperature sensor 36 located along common pneumatic line 46 are recorded by control unit 50 over multiple points in time until the pneumatic pressure reaches specified or commanded value, e.g., five psig, on the pneumatic side of flexible sheet 112 at pump actuation chamber 26. Since fluid side valves 28a to 28e are closed, fluid side pressure follows air side pressure with time.

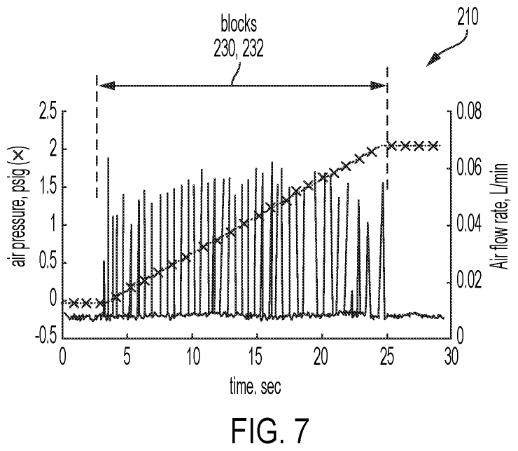

FIG. 7 illustrates that the operation of blocks 230 and 232 occurs from about three seconds to about twenty-five seconds. Here, the pressurization begins at zero and ends with about two psig. During this period, the air flow fluctuates up and down, which is recorded by air flow sensor 32 along with the pneumatic pressure recorded by pressure sensor 34. Air temperature is also recorded by temperature sensor 36. At about twenty-five seconds, the pneumatic pressure flattens at about two psig and air flow drops to zero.

At block 234, control unit 50 then performs a before and after calculation to approximately determine how much fresh or used PD fluid has been pulled into pump chamber 110. In an embodiment, two algorithms or equations are used, wherein the first algorithm or equation is used twice, before and after each collection of flow sensor 32, pressure sensor 34 and temperature sensor 36 data. The second equation then determines the difference between the outcomes of the initial and final (before and after) uses of the first equation. The first equation used for the initial five psig application (or two psig in FIGS. 5 and 7) is in one embodiment:

$$\begin{matrix}\text{?} = \frac{\{ [ \sum {P}_{i} ( t ) \frac{{f}_{i} ( t ) Δ t}{R \text{?} ( t )} ] R \text{?} \}}{Δ {P}_{i}}\end{matrix} ,$$

$$\text{?} \text{indicates text missing or illegible when filed}$$

which is again derived from the Ideal Gas Law, and wherein R is a constant, f for a given point in time is the measured flowrate, P for the given point in time is the measured pressure and T for the given point in time is the measured temperature (for the initial five psig (or two psig) application). Vi is the initial pneumatic side air volume in pump actuation chamber 26 before drawing fresh or used PD fluid into the fluid side of the pump chamber at blocks 224 and 226. The first equation used for the final five psig application is then:

$$\begin{matrix}\text{?} = \frac{\{ [ \sum {P}_{f} ( t ) \frac{{f}_{f} ( t ) Δ t}{{RT}_{f} ( t )} ] R \text{?} \}}{Δ {P}_{f}}\end{matrix} ,$$

$$\text{?} \text{indicates text missing or illegible when filed}$$

wherein R is a constant, f for a given point in time is the measured flowrate, P for the given point in time is the measured pressure and T for the given point in time is the measured temperature (for the final five psig (or two psig) application). Vf is the final pneumatic side air volume in pump actuation chamber 26 after the fluid draw at blocks 224 and 226. The second equation used for determining (approximating) how much fresh or used PD fluid is drawn into pump chamber 110 may then be:

$$Δ V = Vf - Vi ,$$

wherein ΔV is the amount of fresh or used PD fluid that has been drawn into pump chamber 110. At block 236, control unit 50 may then use the amount drawn for a subsequent discharge stroke to know how much (approximately) fresh or used PD fluid is available to deliver. After block 236, method 210 returns to discharge method 150 as illustrated in FIG. 4.

The following paragraphs discuss using the methodology of system 10 of the present disclosure for occlusion detection, patient or bag empty detection and air in fluid detection. For the first two (occlusion/empty) there are multiple ways for making such detections. In a first way, during a delivery stroke or suction stroke, control unit 50 analyzes the air flowrate profile resulting from the output of flow sensor 32. Control unit 50 detecting no change in air flowrate determines a complete occlusion or bag/patient completely empty. Control unit 50 detecting a lower rate of decay of air flowrate determines a partial occlusion or bag/patient near empty. In a second way, control unit 50 makes the determinations based on a measured suction or delivery fluid volume by movement of flexible sheet 112 compared against an expected suction or delivery fluid volume, wherein the expected volume is based on a volume defined by disposable pump chamber 110. No actual volume pumped indicates a full occlusion or full empty condition to control unit 50. A partial but less than expected volume pump indicates a partial occlusion or empty condition to control unit 50. In a third way, control unit 50 makes determinations based on a difference in the volume measured between a suction stroke and a delivery stroke. The third way is used by control unit 50 in an embodiment to determine between an occlusion and an empty condition, which has been detected by control unit 50 via one of the first two ways, here attempting to push back to the same line upon which the suction stroke is made. If resistance is still found in the delivery stroke to the same line, then control unit 50 determines that an occlusion has occurred. If the resistance seen in the suction stroke is no longer present in the delivery stroke to the same line (here may need to pull fluid from a different source for the delivery stroke), then control unit 50 determines that empty condition has occurred.

The draw approximation and subsequent accurate discharge determination may be used to detect a full or partial occlusion. The draw approximation (method 210) during a partial or full occlusion would show that very little or no fresh or used PD has entered pump chamber 110 from the patient or from a PD fluid container or bag 104a to 104c. The accurate discharge determination (method 150, here attempting to pump to the same line as the attempted draw) would show an inability (little or none) to push back fresh or used PD fluid to the patient or a drain container or house drain 104d. In particular, during the discharge of PD fluid, control unit 50 in an embodiment looks at the resulting flowrate profile to determine if a partial or complete occlusion has occurred. Control unit 50 alternatively or additionally determines an occlusion based on a measured PD fluid discharge volume compared against an expected PD fluid discharge volume. It is contemplated for control unit 50 to verify a partial or complete occlusion by causing the application of different air pressures at the pneumatic side of the flexible sheet. The partial or complete occlusion should be detected by control unit 50 at each varied air pressure as confirmation. Higher pressures may be limited by control unit 50 to PD fluid supply lines 106a to 106c and drain line 106d, so that the patient does not see the higher pressures along patient line 106e.

The draw approximation and subsequent accurate discharge determination may also be used to detect a patient empty condition or a bag empty condition. The draw approximation performed by control unit 50 during a patient empty condition would show that very little used PD fluid has been pulled from the patient. The draw approximation performed by control unit 50 during a container or bag empty condition would show that very little fresh PD fluid has been pulled from the PD fluid container or bag 104a to 104c. The accurate discharge determination performed by control unit 50, which causes air pump 40 to attempt to pump to drain container 104d or house drain (for patient empty condition) or to patient line 106e (for bag empty condition), would however show that a small amount of (i) effluent is able to be pushed to a house drain or drain container 104d (for patient empty), or (ii) fresh PD fluid is able to be pushed to the patient (for bag empty).

Control unit 50 may alternatively or additionally be configured to detect air during the draw approximation by observing a higher than normal Vi or Vf determination. In particular, control unit 50 may determine the presence of air in disposable set 100 based on an absolute volume measurement during a draw stroke. The presence of air results in a higher Vi or Vf determination than typical (lower fluid volume), which may be due to a partial occlusion in the fluid suction line 106a to 106c or 106e (in the case of Vf) or one or more air bubble in pump chamber 110 of disposable set 100 (for both Vi and Vf). It is contemplated for control unit 50 to confirm the presence of air by causing the application of different air pressures to determine Vi or Vf as applicable. Here, the presence of air causes a variation in the Vi or Vf measurement/calculation as a function of positive pressure for each of the different pressures applied, which may be observed for verification.

It should be understood that various changes and modifications to the presently preferred embodiments described herein will be apparent to those skilled in the art. For example, separate positive and negative air pumps may be used to supply positive and negative air pressure instead of single air pump 40. In another example, PD fluid heater 30 could be located remote from cycler 20. Such changes and modifications can be made without departing from the spirit and scope of the present subject matter and without diminishing its intended advantages. It is therefore intended that such changes and modifications be covered by the appended claims.

1: A peritoneal dialysis (“PD”) system comprising:
a disposable set including a pump chamber having a flexible sheet, a pneumatic side of the flexible sheet positioned and arranged during operation to receive pneumatic pressure; and
a cycler including
an air pump for delivering positive and negative pneumatic pressure to the pump chamber,
a pneumatic delivery line and a pneumatic suction line extending from the air pump, the pneumatic delivery line and the pneumatic suction line in selective pneumatic communication with the pneumatic side of the flexible sheet,
an air flow sensor,
a pneumatic pressure sensor,
a plurality of fluid valves, and
a control unit configured to integrate outputs from the air flow sensor and the pneumatic pressure sensor over time to determine an amount of fresh or used PD fluid discharged from the pump chamber under positive pneumatic pressure and via an open one of the plurality of fluid valves.

2-3. (canceled)

4: The PD system of claim 1, wherein the cycler includes a pump actuation chamber covered in operation by the pneumatic side of the flexible sheet, the pneumatic delivery line and the pneumatic suction line in selective pneumatic communication with the pump actuation chamber.

5: The PD system of claim 1, further comprising a pneumatic rigid plastic shell connected to the pump chamber so as to seal the flexible sheet between the pneumatic rigid plastic shell and the pump chamber,
wherein the pneumatic delivery line and the pneumatic suction line are in selective pneumatic communication with a port provided by the pneumatic rigid plastic shell.

6: The PD system of claim 1, wherein the pneumatic delivery line and the pneumatic suction line extend to a common pneumatic line,
wherein the air flow sensor and the pneumatic pressure sensor are in operable communication with the common pneumatic line.

7: The PD system of claim 6, further comprising an air temperature sensor in operable communication with the common pneumatic line, the control unit further configured to integrate outputs from the air temperature sensor to determine the amount of fresh or used PD fluid that has been discharged from the pump chamber.

8: The PD system of claim 1, further comprising a first pneumatic valve located along the delivery line and a second pneumatic valve located along the pneumatic suction line, the first and second pneumatic valves providing selective pneumatic communication with the pneumatic side of the flexible sheet.

9: The PD system of claim 1, wherein the air pump is pneumatically coupled to positive and negative pneumatic storage vessels.

10: The PD system of claim 1, wherein the control unit is configured to integrate outputs from the air flow sensor and the pneumatic pressure sensor over time using an algorithm derived from the Ideal Gas Law.

11: The PD system of claim 1, wherein the control unit is configured to integrate outputs from the air flow sensor and the pneumatic pressure sensor over time for (i) an initial pneumatic pressurization of the pump chamber to determine an initial chamber volume and (ii) a final pneumatic pressurization of the pump chamber to determine a final chamber volume, the control unit further configured to determine an amount of fresh or used PD fluid drawn into the pump chamber by subtracting the initial chamber volume from the final chamber volume.

12: The PD system of claim 11, wherein the control unit is configured to cause negative pneumatic pressure to be applied to the pump chamber with one of the plurality of fluid valves opened between the initial pneumatic pressurization and the final pneumatic pressurization.

13: The PD system of claim 11, wherein the control unit is configured to cause at least one pneumatic line to be vented prior to each of the initial pneumatic pressurization and the final pneumatic pressurization.

14: The PD system of claim 11, wherein the control unit is further configured to use (a) the output from the air flow sensor or (b) the amount of fresh or used PD fluid drawn into the pump chamber to at least partially determine at least one of (i) a partial or full line occlusion, (ii) a PD fluid container or patient empty condition, or (iii) a presence of air.

15: The PD system of claim 14, wherein the control unit is configured to use the amount of fresh or used PD fluid discharged from the pump chamber to confirm or fully determine at least one of (i) the partial or full line occlusion, or (ii) the PD fluid container or patient empty condition.

16: The PD system of claim 14, wherein the control unit is configured to attempt to have fresh or used PD fluid pushed through a same line from which fresh or used PD fluid has just previously been subjected to a fresh or used PD fluid draw to determine between (i) the partial or full line occlusion, and (ii) the PD fluid container or patient empty condition.

17: The PD system of claim 1, which further includes an air temperature sensor, and wherein the control unit is configured to determine the amount of fresh or used PD fluid discharged from the pump chamber according to the equation:
$$Δ V = \frac{[ \sum P ( t ) \frac{f ( t ) Δ t}{RT ( t )} ]}{P} RT ,$$
wherein R is a constant, f is measured air flowrate, P is measured air pressure and T is measured temperature.

18: A peritoneal dialysis (“PD”) system comprising:
a disposable set including a pump chamber having a flexible sheet, a pneumatic side of the flexible sheet positioned and arranged during operation to receive pneumatic pressure; and
a cycler including
at least one source of positive and negative pneumatic pressure for delivering pneumatic pressure to the pump chamber,
an air flow sensor,
a pneumatic pressure sensor,
a plurality of fluid valves, and
a control unit configured to:
integrate outputs from the air flow sensor and the pneumatic pressure sensor over time for (i) an initial pneumatic pressurization of the pump chamber to determine an initial chamber volume and (ii) a final pneumatic pressurization of the pump chamber to determine a final chamber volume, wherein the control unit causes negative pneumatic pressure to be applied to the pump chamber with one of the plurality of fluid valves opened between the initial pneumatic pressurization and the final pneumatic pressurization, and
determine an amount of fresh or used PD fluid drawn into the pump chamber by subtracting the initial chamber volume from the final chamber volume.

19. (canceled)

20: The PD system of claim 18, wherein the control unit is configured to cause at least one pneumatic line to be vented prior to each of the initial pneumatic pressurization and the final pneumatic pressurization.

21: The PD system of claim 18, wherein the control unit is further configured to use (a) the output from the air flow sensor or (b) the amount of fresh or used PD fluid drawn into the pump chamber to at least partially determine at least one of (i) a partial or full line occlusion, (ii) a PD fluid container or patient empty condition, or (iii) a presence of air.

22: The PD system of claim 21, wherein the control unit is configured to attempt to have fresh or used PD fluid pushed through a same line from which fresh or used PD fluid has just previously been subjected to a fresh or used PD fluid draw to determine between (i) the partial or full line occlusion, and (ii) the PD fluid container or patient empty condition.

23: The PD system of claim 18, which further includes an air temperature sensor, and wherein the control unit is configured to integrate outputs from the air flow sensor, the pneumatic pressure sensor and the air temperature sensor over time for determining the an amount of fresh or used PD fluid drawn into the pump chamber according to the equations:
$$Δ V = Vf - Vi , \qquad ( i )$$
$$\begin{matrix}\text{?} = \frac{\{ [ \sum {P}_{f} ( t ) \frac{{f}_{f} ( t ) Δ t}{{RT}_{f} ( t )} ] R \text{?} \}}{Δ {P}_{f}}\end{matrix} , \qquad ( ii )$$
$$and$$
$$\begin{matrix}{V}_{i} = \frac{\{ [ \sum {P}_{i} ( t ) \frac{{f}_{i} ( t ) Δ t}{R \text{?} ( t )} ] R \text{?} \}}{Δ {P}_{i}}\end{matrix} , \qquad ( iii )$$
$$\text{?} \text{indicates text missing or illegible when filed}$$
wherein R is a constant, f is measured air flowrate, P is measured air pressure and T is measured temperature.
